(19) United States
(12) Patent Application Publication
(10) Pub. No.: US 2004/0181821 A1
(43) Pub. Date: Sep. 16, 2004

(54) siRNA research tool kit

(76) Inventors: Jiadong Zhou, Vista, CA (US); Kenji Matsumoto, San Diego, CA (US); Lei Yu, Carlsbad, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/789,590

(22) Filed: Feb. 27, 2004

Related U.S. Application Data
(60) Provisional application No. 60/455,220, filed on Mar. 14, 2003.

(51) Int. Cl. A01K 67/00; C12Q 1/68; C12N 15/82; C07H 21/04

(52) U.S. Cl. 800/9; 536/23.1; 435/6

(57) ABSTRACT

A kit is disclosed for gene functional studies which allows users to produce siRNA via siRNA expression cassettes, efficiently introduce the siRNA expression cassette into cultured mammalian cells, evaluate the transfection efficiency, and evaluate the siRNA synthetic efficiency. The kit includes a siRNA synthetic system, a transfection reagent(s), a PCR primer with a specific fluorescent dye tag for tracking down the siRNA delivery pathway and a reporter gene cassette such as GFP gene expression cassette, for easily selecting the cells that are successfully transfected with the siRNA in the whole cell pool.

### RELATED APPLICATIONS

This application claims the priority of U.S. Provisional application No. 60/455,220, filed Mar. 14, 2003, which is incorporated herein by reference in its entirety.

### BACKGROUND OF THE INVENTION

1. Field of the Invention

In one embodiment, the present invention relates to the introduction of a fluorescence tag into an siRNA expression system for tracking the delivery pathway of an siRNA expression cassette. In a preferred embodiment, Green Fluorescent Protein (GFP) may be used as a reporter gene in a synthetic cassette for indicating siRNA synthesis in nuclei of the transfected cells.

2. Description of the Related Art

Short interfering RNA (siRNA) is a special case of RNA interference (RNAi). RNAi was first discovered in 1998 by Andrew Fire's group in the Carnegie Institution of Washington. When the authors introduced double stranded RNA (dsRNA) corresponding to a sense and antisense sequence of an endogenous mRNA into C. elegans, either by feeding or injection, the cognate mRNA was degraded and the expression of the gene was silenced. It quickly became clear that RNAi is present in many other organisms (the fruit fly Drosophila, certain protozoa, plants), and that the process is ancient. In fact it is the oldest and most ubiquitous antiviral system, appearing before the divergence of plants and animals (Sharp, P. A. (2001) RNA interference-2001. Genes Dev. 15, 485-490).

In mammalian systems, RNAi becomes more complicated, because of the existence of a non-specific pathway, where long dsRNA activates a protein kinase, PKR, and 2′,5′ oligoadenylate synthetase (2′,5′-AS). This results in the shut down of protein synthesis and degradation of all mRNAs. This mechanism is part of the mammalian cell response to viral infection, and its existence masks any RNA interference effect. It was later discovered that short dsRNA (19-25 base pair) could bypass the more global, non-specific response, and allow the observance of RNA interference effects. Because of its size, this type of RNAi effect is called short interfering RNA (siRNA; Elbashir, S. M. et al. (2001) Duplexes of 21-nucleotide RNAs mediate RNA interference in cultured mammalian cells. Nature 411: 494-498).

From a researcher's standpoint, siRNA is an extremely valuable tool, both for research and medicine. Because siRNA (and RNAi in general) is based on nucleotide-nucleotide base-pairing interactions, it can be tailored to target any gene of interest, as long as at least a portion of the sequence is known, to knock out the expression of that gene, and study the resulting phenotype to infer the function(s) of the gene. This eliminates the need of mutating the genetic material in an organism, which is generally time consuming and costly. siRNA can be used to target disease genes. In fact, due to its high sequence specificity, the sequence difference between the disease gene and the wild type gene can be exploited to target only the altered, disease causing gene, without the danger of affecting normal genes. Applications towards HIV and cancer treatment have been reported (Qin et al. (2003) Inhibiting HIV-1 infection in human T cells by lentiviral-mediated delivery of small interfering RNA against CCR5. PNAS 100, 183-188; Wilda et al. (2002) Killing of leukemic cells with a BCR/ABL fusion gene by RNA interference (RNAi). Oncogene 21, 5716-5724).

Chemically synthesized siRNA was used in the original siRNA study (Elbashir et al., 2001). Chemical synthesis of siRNA is straightforward, and is routinely done using a conventional DNA/RNA synthesizer. Two strands of RNA need to be synthesized individually, and annealed before use. Because RNA is labile, appropriately protected ribonucleoside phosphoramidites need to be used during synthesis. More often, siRNAs are obtained from commercial RNA oligonucleotide synthesis suppliers. Cost is generally high (>$300/pair, as compared to ˜$ 10/pair for DNA oligos of the same length).

More recently, siRNAs expressed from a DNA template have been shown to be as effective in silencing gene expression as chemically synthesized siRNAs (Yu, J. Y. et al. (2002) RNA interference by expression of short-interfering RNAs and hairpin RNAs in mammalian cells. PNAS 99, 6047-6052; Paddison, P. J. et al. (2002) Short hairpin RNAs (shRNAs) induce sequence-specific silencing in mammalian cells. Genes Dev. 16, 948-958; Lee, N. S. et al. (2002) Expression of small interfering RNAs targeted against HIV-1 rev transcripts in human cells. Nature Biotechnology 20, 500-505; Brummelkamp, T. R. et al. (2002) A system for stable expression of short interfering RNAs in mammalian cells. Science 296, 550-553; Paul, C. P. et al. (2002) Effective expression of small interfering RNA in human cells. Nature Biotechnology 20, 505-508; Miyagishi, M. and Taira, K. (2002) U6 promoter driven siRNAs with four uridine 3′ overhangs efficiently suppress targeted gene expression in mammalian cells. Nature Biotechnology 20, 497-500; Sui, G. et al. (2002) A DNA vector-based RNAi technology to suppress gene expression in mammalian cells. PNAS 99, 5515-5520; Zeng, Y. et al. (2002) Both natural and designed microRNAs can inhibit the expression of cognate mRNAs when expressed in human cells. Mol. Cell 9, 1-20; McManus, M. T. et al. (2002) Gene silencing using micro-RNA designed hairpins. RNA 8, 842-850). This method is more versatile, more cost effective and is now widely used.

For biological siRNA synthesis, the siRNA expression cassette has to be delivered to the nuclei of the cells. Therefore, transfection is an important step in gene knock down studies with the biological siRNA synthetic strategy. An efficient and reliable transfection reagent is needed. The ability to track the siRNA expression cassette in the cells of interest during the transfection process is useful for data analysis in such experiments.

Chemically synthesized siRNA is commercially available from companies such as Dharmacon and Ambion. Allele provides a product (LineSilence™) that provides a stable DNA-based method for RNA interference (RNAi), using U6 promoter. Ambion provides a plasmid based siRNA expression, also using U6 promoter. BioCarta provides a plasmid-based system to generate siRNA for gene knockdown which includes a transfection reagent in the kit (GeneSuppressor™ RNA Interference Kit). InvivoGen provides a H1 promoter based psiRNA system.

Currently no product is available for evaluation of both cellular and nuclear delivery. A problem with currently available technology is that it is difficult to pinpoint the problem when an experiment fails, without resorting to additional reagents and equipment.

Preferred embodiments of the present invention allow the user to evaluate the status of an experiment in vivo. A fluorescence label is included on the PCR primer. The label is used to track the cellular delivery of the siRNA expression cassette. Additionally, a GFP expression unit is included in the siRNA expression cassette. GFP expression is used as an indicator for nuclear delivery of the siRNA expression cassette. Consequently, both cellular and nuclear delivery can be conveniently monitored. The system we have built creates an “One-stop-shopping” option for siRNA users. Compared to existing products, it not only provide siRNA synthesis and siRNA delivery tools, but also allows the user to monitor the progress of the experiment and troubleshoot any problems.

### SUMMARY OF THE INVENTION

Some embodiments of the invention pertain to a siRNA expression cassette which includes a promoter; a sequence encoding double stranded (ds) RNA operatively linked to the promoter; and a sequence encoding a nuclear delivery indicator. Preferably, the ds RNA is about 19-25 base pairs. In preferred embodiments, the sequence encoding the ds RNA and the sequence encoding the nuclear delivery indicator are on the same plasmid or vector. Preferably, the promoter operably linked to the sequence encoding the ds RNA is a U6 or H1 promoter.

In some preferred embodiments, the siRNA expression cassette also includes one or more indicators of cellular delivery of the expression cassette. Preferably, the indicator of cellular delivery is a fluorescent dye, a quantum dot or an imaging molecule for non-invasive diagnosis. Preferably, the non-invasive diagnosis is selected from NMR, MRI, CT, and PET scanner.

Preferably, the siRNA expression cassette expresses small RNA in mammalian cells.

In preferred embodiments, the sequence encoding a nuclear delivery indicator is a sequence for GFP or luciferase operably linked to a promoter. Preferably, the promoter is tissue specific.

Another embodiment of the invention is directed to a kit for in vitro and/or in vivo gene knock down studies at RNA level which includes the following parts:

1) a short interfering RNA (siRNA) expression cassette;

2) a reporter gene cassette that exhibits reporter gene expression if it is successfully delivered to, and expressed in, the nuclei of a eukaryotic cell; and

3) a transfection reagent that is able to efficiently deliver the siRNA expression cassette and the reporter gene cassette into the eukaryotic cells.

In preferred embodiments, the siRNA expression cassette and the reporter gene cassette are on the same plasmid or vector. More preferably, the reporter gene cassette is adjacent to a DNA fragment of the siRNA expression cassette. More preferably, the interference RNA expression cassette and the report gene synthesis cassette are circular or linear.

Preferably, the siRNA expression cassette is driven by a U6 promoter or a H1 promoter and expresses small RNA in mammalian cells. Preferably, the siRNA expression cassette expression is directed to non-specific expression, tissue-specific expression or cell-specific expression.

Preferably, the siRNA expression cassette also includes an intracellular traffic marker. More preferably, the intracellular traffic marker is a fluorescent dye, a quantum dot or an imaging molecule for non-invasive diagnosis. The non-invasive diagnosis may be NMR, MRI, CT, or PET scanner.

In preferred embodiments, the reporter gene cassette is a fluorescent protein gene expression cassette operably linked to a promoter. More preferably, the promoter confers tissue specific expression. More preferably, the fluorescent protein gene expression cassette encodes a green fluorescent protein gene.

Preferably, the transfection agent is selected from the group including lipid based transfection reagents with or without cationic groups, polymer based transfection reagents with or without cationic groups, lipid-polymer-based transfection reagents with or without cationic groups, polysaccharide-based transfection reagents with or without cationic groups, and peptide-based transfection reagents with or without cationic groups. More preferably, the transfection reagent further includes a cell culture reagent. Yet more preferably, the cell culture reagent is a cytoreductive reagent, cell attach reagent, cell growing reagent, or cell inhibiting reagent.

More preferably, the transfection reagent is a gene delivery enhancer or a targeting molecule. Yet more preferably, the gene delivery enhancer is an endosomalytic reagent or a nuclear localization element.

More preferably the targeting molecule is a peptide, a protein, an antibody or its related fragment, a sugar, or a synthetic molecule.

Further aspects, features and advantages of this invention will become apparent from the detailed description of the preferred embodiments which follow.

### BRIEF DESCRIPTION OF THE DRAWINGS

These and other feature of this invention will now be described with reference to the drawings of preferred embodiments which are intended to illustrate and not to limit the invention.

FIG. 1 shows the restriction digest pattern with XhoI and SacI (on 1% agarose gel) of cloned Human U6 and H1, with pBSKS as vector control.

FIG. 2A) shows the overall structure of an siRNA cassette according to the invention. FIG. 2B) shows the production of the cassette by PCR. A two step PCR is shown in 2B a). A one step PCR is shown in FIG. 2B b). FIG. 2B c) shows PCR of two strands which are then annealed.

FIG. 3 shows the increased GFP signal with increased amount of siRNA cassette (which contains GFP reporter) used in transfection.

FIG. 4 shows the increased target gene inhibition with increased amount of siRNA cassette (which contains GFP reporter) used in transfection. Three different ratios of transfection agent: DNA are shown. “RLU” signifies relative lighting unit.

FIG. 5 shows the effect of siRNA cassette (targeting VEGF) on breast cancer tumor in mouse model. siRNA R is reverse orientation version of the siRNA cassette.

FIG. 6 shows more detailed analysis of the effect of siRNA cassette on breast cancer tumor in mouse model. FIG. 6A shows the tumor weight in grams for the described treatments. FIG. 6B shows the microvascular density after one month treatment. FIG. 6C shows tumor VEGF level after one month for the described treatments.

### DETAILED DESCRIPTION OF THE PREFERRED EMBODIMENT

In some embodiments, the invention is directed to a siRNA expression cassette which includes both a sequence to express the siRNA and a nuclear delivery indicator. The sequence to express the siRNA includes a sequence which encodes a double stranded RNA. Preferably, the double stranded RNA is less than 100 bases. More preferably, the double stranded RNA is less than 50 bases. Yet more preferably, the double stranded RNA is less than 25 bases. Most preferably, the double stranded RNA is 19-25 base pairs.

The sequence encoding the double stranded RNA is operably linked to a promoter. In preferred embodiments, the promoter is a U6 or H1 promoter, or any other promoter that is capable of driving the expression of short RNA.

The promoter can be a combination of two or more promoters. The promoter can be a combination between a promoter and other regulatory elements, such as tissue or cell specificity determination signal.

The siRNA expression cassette also includes at least one nuclear delivery indicator. The nuclear delivery indicator provides an indication that the siRNA has been delivered to the nucleus of the transfected cell. In preferred embodiments, the nuclear delivery indicator is an expressible gene linked to a promoter which provides a signal when expressed. In preferred embodiments, the nuclear delivery indicator is a reporter gene encoding a fluorescent protein (such as Green Fluorescent Protein, or GFP), luciferase, β-glucuronidase or β-galactosidase. Most preferably, the nuclear delivery indicator is a fluorescent protein operably linked to a promoter.

In some embodiments, the siRNA expression cassette includes an indicator of cellular delivery or intracellular traffic marker. In some embodiments, the indicator of cellular delivery is a label included on the cassette. In preferred embodiments, the label is a fluorescent label, a quantum dot or an imaging molecule that is either covalently or non-covalently attached to the siRNA expression cassette. Imaging molecules such as those adapted for NMR, MRI, CT and PET scanner may be adapted for use in the siRNA expression cassette described herein.

The siRNA expression cassette may be adapted for any cell type. For example, the siRNA expression cassette may be used in any type of eukaryotic cell, including plant cells and insect cells. In preferred embodiments, the siRNA expression cassette is adapted for use in mammalian cells.

In a preferred embodiment, the siRNA expression cassette is included in a composition or kit for in vitro and/or in vivo gene knock down studies at the RNA level. In preferred embodiments, the kit includes the following parts: 1) a short interfering RNA (siRNA) expression cassette operably linked to a promoter with or without an intracellular traffic marker; 2) a nuclear delivery molecule such as a reporter gene cassette operably linked to a second promoter that is used to indicate whether it has been successfully delivered to and expressed in the nuclei of a eukaryotic cell; and 3) a transfection reagent that is able to efficiently deliver the siRNA expression cassette and the reporter gene cassette into the eukaryotic cells; and optionally, 4) reagents needed for PCR (except the siRNA sequence containing primer); and 5) reagents and material needed for PCR product purification (except ethanol, centrifuge, eppendorf tubes)

The kit is useful for any type of eukaryotic cell including plant and insect cells. In preferred embodiments, the cells are mammalian cells.

In a preferred embodiment, the siRNA expression cassette is driven by a promoter and expresses small RNA in eukaryotic cells. Preferably, the RNA is less than 100 bases. More preferably, the RNA is less than 50 bases. Yet more preferably, the RNA is less than 25 bases. Most preferably, the RNA is 19-25 base pairs. Any promoter that expresses small RNA in eukaryotic cells may be used. Preferably the promoter is a U6 or H1 promoter. The siRNA expression cassette may direct non-specific expression, tissue-specific expression or cell-specific expression.

In some preferred embodiments, the siRNA expression cassette includes an intracellular traffic marker which is an indicator of cellular delivery of the expression cassette. Preferably, the intracellular traffic marker may be a fluorescent dye, a quantum dot, or an imaging molecule for non-invasive diagnosis. The non-invasive diagnosis is generally one of NMR, MRI, CT, or PET scanner, for example. The intracellular delivery marker may be covalently or non-covalently attached to the siRNA expression cassette.

The reporter gene may be any reporter gene known in the art including β-galactosidase, β-glucuronidase, luciferase, and fluorescent protein. In a preferred embodiment, the reporter gene cassette is a fluorescent protein gene expression cassette operably linked to a promoter. In some preferred embodiments, the promoter is tissue specific. In most preferred embodiments, the fluorescent protein gene expression cassette includes a green fluorescent protein gene or a gene encoding luciferase.

Preferably, the siRNA cassette and reporter gene cassette are on the same plasmid or vector. In some preferred embodiments, the reporter gene cassette is adjacent to the siRNA expression cassette on a DNA fragment. In preferred embodiments, the siRNA expression cassette and the reporter gene cassette may be circular or linear.

The siRNA expression cassette allows the progress of an experiment, such as a gene knock-out experiment to be evaluated. The evaluation may be performed by any means known in the art including withdrawal of sample at specific time points or the experiment may be evaluated in real time. For example, if fluorescent protein is used as the reporter gene, the progress of the experiment may be followed using a fluorescence microscope or fluorometer. The activity of luciferase can be detected by a luminometer, and the blue product catalyzed by P-galactosidase can be observed under microscope or determined by a microplate reader. One of skill in the art is familiar with how these reporters function.

Alternatively, the nuclear delivery indicator may be delivered according to methods well known to those skilled in the art, such as detecting immunofluorescence or enzyme immunocytochemistry, autoradiography, or in situ hybridization. If immunofluorescence is used to detect expression of an encoded protein, a fluorescently labeled antibody that binds the target protein is used (e.g., added to a slide under conditions suitable for binding of the antibody to the protein). Cells containing the protein are then identified by detecting a fluorescent signal. However, the identification method depends on the properties of the nuclear delivery indicator.

Preferably, the transfection reagent shows efficient nucleic acid delivery. The transfection agent may be a lipid based transfection reagent with or without cationic groups, a polymer based transfection reagent with or without cationic groups, a lipid-polymer-based transfection reagent with or without cationic groups, a polysaccharide-based transfection reagent with or without cationic groups, or a peptide-based transfection reagent with or without cationic groups. The transfection reagent optionally includes a cell culture reagent. The cell culture reagent may be a cytoreductive reagent, a cell attachment reagent, a cell growth reagent, or a cell inhibition reagent.

The transfection reagent may include a gene delivery enhancer or a targeting molecule. Gene delivery enhancers include but are not limited to an endosomalytic reagent and a nuclear localization element. The transfection reagent may include a targeting molecule which may be a peptide, a protein, an antibody or its related fragment, a sugar, or a synthetic molecule, for example. Transfection reagents that are useful in some embodiments of the invention are taught in U.S. Application No. 60/507,161, filed Sep. 29, 2003 and U.S. application Ser. No. 10/375,705, filed Feb. 25, 2003, both of which are incorporated herein by reference.

### EXAMPLES

### Example 1

### Preparation of siRNA Expression Cassette

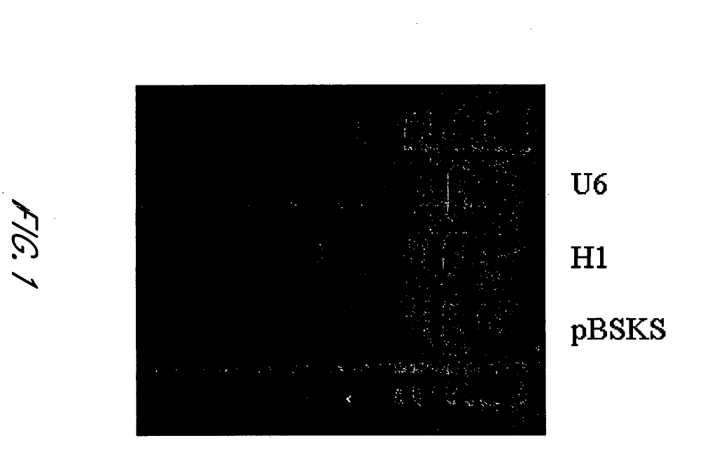

Human H1 and U6 genes (including the promoter region) were cloned (FIG. 1), giving rise to pBSKSH1 and pBSKSU6, respectively. siRNA expression cassette is derived by PCR, using promoter upstream primer together with a downstream primer containing siRNA sequence. The expression of siRNA targeting either GFP or luciferase has been verified.

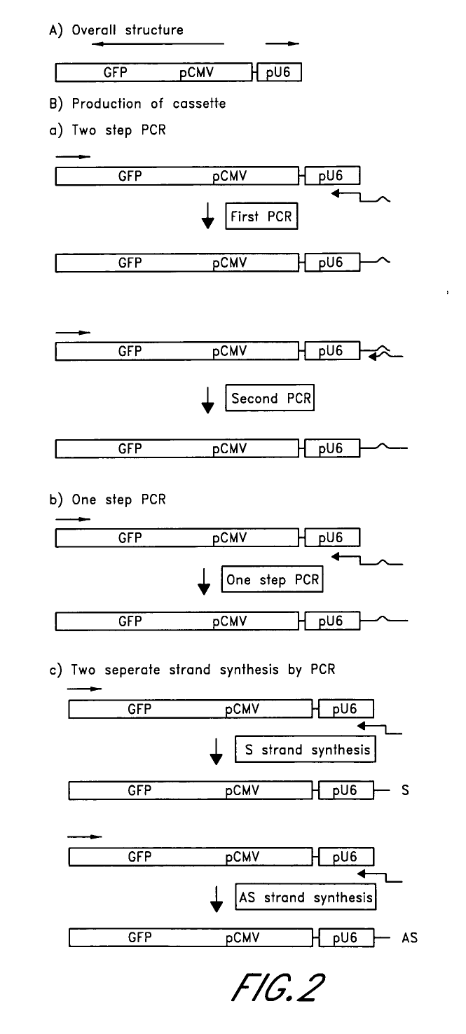

In order to be able to evaluate the nuclear delivery of the siRNA expression cassette, a GFP expression cassette was included in the siRNA expression cassette (FIG. 2, A). Using pBI-eGFP as template (BD Biosciences Clontech), eGFP was amplified with primers eGFP_S_HindIII & eGFP_AS_XbaI. The PCR fragment was digested with HindIII/XbaI, and ligated into the pcDNA3.1/Hygro(+) vector digested with the same enzymes. This gave rise to pcDNA3.1_Hygro(+)_GFP_HindIII_XbaI. Because of the presence of the hygromycin resistance gene, this plasmid also makes it possible to set up a cell line which can stably express GFP.

Using primers CMV_S_HindIIIEcoRI and BGHpA_AS_KpnIXhoI, a fragment was amplified by PCR that included the CMV promoter, the eGFP gene, and the BGH poly A region. The amplified fragment was digested with EcoRI and KpnI, and ligated into pBSKSU6 digested with the same enzymes. The resulting plasmid is pBSKS_r_CMVGFP_U6, which now contains a CMV-eGFP cassette in the U6-based siRNA expression vector. When PCR is carried out using BGH polyA upstream primer and U6 promoter downstream primer (containing the siRNA sequence), the PCR product (expression cassette) contains both the eGFP cassette and the U6-siRNA cassette. This allows the expression of siRNA and the evaluation of the nuclear delivery of the siRNA expression cassette.

FIG. 2 B describes three different ways of producing the cassette by means of PCR. The first two belong to the category of producing a single cassette that will express a hairpin-structured RNA, which will be cleaved in a cell to become siRNA. The procedures for FIG. 2B a) and b) were performed according to the instructions in the Ambion Silencer™ Express Instruction Manual. Basically, for the two step method of FIG. 2B a), one oligonucleotide was designed with its 3′ end complementary to the 3′ end of a RNA Pol III promoter element and the 5′ end complementary to a loop and sense strand of the desired siRNA. The second oligonucleotide has a 5′ end encoding the RNA Pol III terminator sequence followed by sequences complementary to the hairpin siRNA antisense strand and loop. The first oligonucleotide is used in a PCR with a RNA Pol III element and RNA Pol III primer. The resulting PCR product is diluted and a fraction is added to a second PCR with the antisense oligonucleotide and RNA Pol III primer. The resulting amplification product is diluted and used to initiate a PCR with the RNA Pol III and terminator primers. The amplification product is column purified. The cassette can also be produced by one PCR, but requiring a longer PCR primer as shown in FIG. 2B b) and as described in the Ambion Silencer™ Express instruction manual.

The third way is to make two different cassettes, each expressing one RNA strand, sense or antisense, as shown in FIG. 2B c). Each cassette is produced by an individual one-step PCR. The two cassettes are introduced into the cell together, each producing one strand of RNA, and these two strands anneal to form siRNA. The two strands will form double stranded siRNA in the cell.

### Example 2

### Correlation Between GFP Signal and siRNA Expression

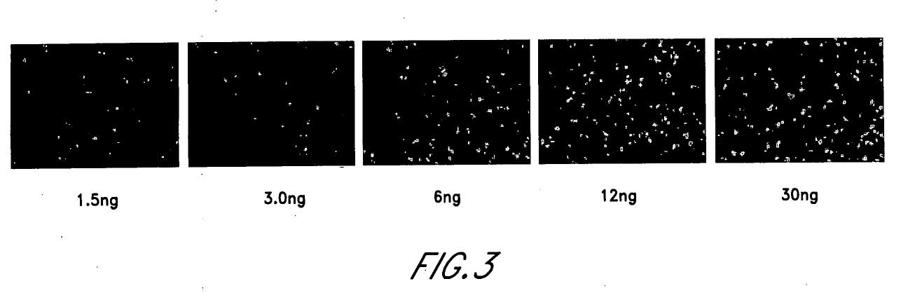

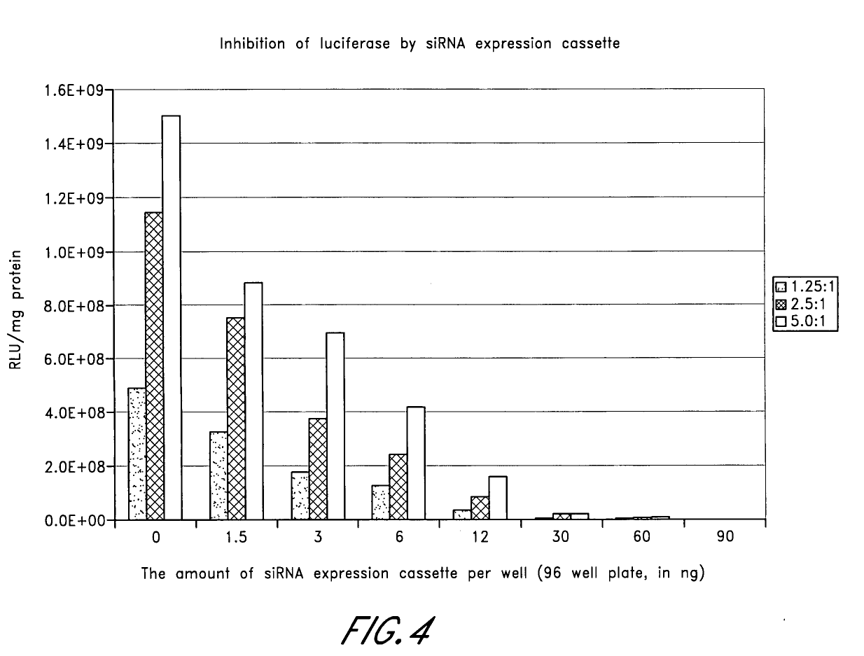

We studied the correlation between GFP signal and siRNA expression level. FIG. 3 shows that strong correlation exists between the GFP signal and the amount of cassette used in transfection. FIG. 4 shows the level of inhibition also correlates well with the cassette amount. Taken together, our results suggest that there is a strong correlation between GFP signal and siRNA expression level, and that we are able to predict the latter using the former. This is one of the useful features of preferred embodiments of the invention.

### Example 3

### Knock-out of VEGF Using siRNA Expression Cassette

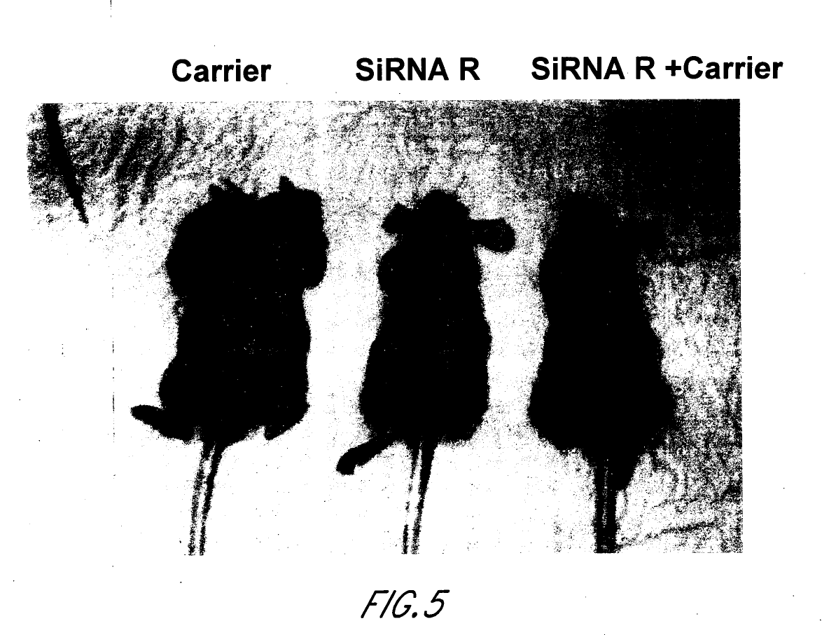

We tested siRNA expression from our siRNA cassette in a mouse breast cancer model. The target we chose was the vascular endothelial growth factor (VEGF) gene. VEGF was chosen because it is a key factor in promoting tumor angiogenesis, which is important in tumor growth, progression, and metastatic dissemination. When the cassette was injected into the pre-planted tumor (with or without gene carrier; repeated treatment during 1 month period), the tumor growth was significantly inhibited compared with gene carrier only control (FIG. 5).

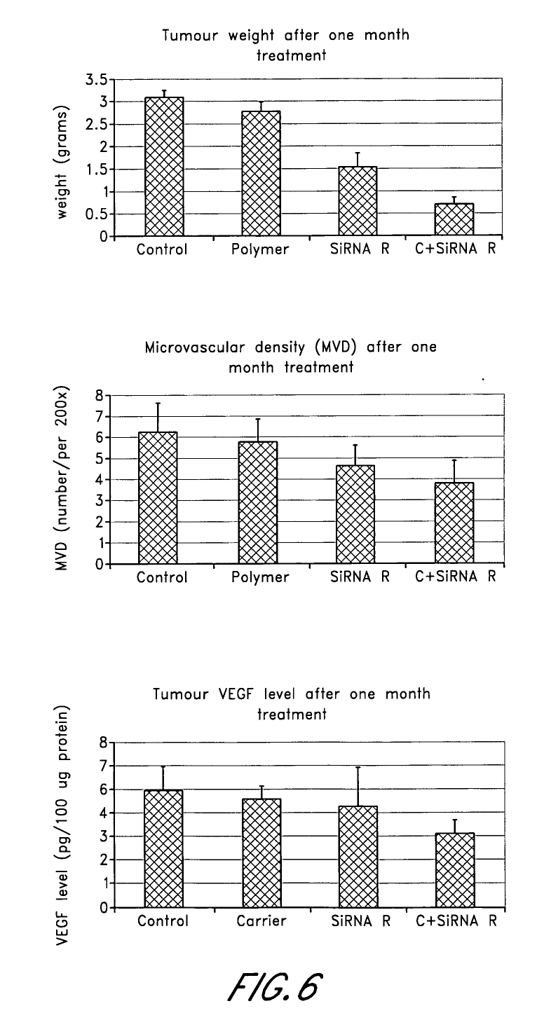

More detailed analysis is shown in FIG. 6. These experiments show the effectiveness of our cassette system to express siRNA in vivo, using an endogenous gene target.

It will be understood by those of skill in the art that numerous and various modifications can be made without departing from the spirit of the present invention. Therefore, it should be clearly understood that the forms of the present invention are illustrative only and are not intended to limit the scope of the present invention.

What is claimed is:

1. A siRNA expression cassette comprising:
a promoter;
a sequence encoding ds RNA of 19-25 base pairs operatively linked to said promoter; and
a sequence encoding a nuclear delivery indicator.

2. The siRNA expression cassette of claim 1 further comprising one or more indicators of cellular delivery of the expression cassette.

3. The siRNA expression cassette of claim 1, wherein the sequence encoding the ds RNA and the sequence encoding the nuclear delivery indicator are on the same plasmid or vector.

4. The siRNA expression cassette of claim 1, wherein the promoter is a U6 or H1 promoter.

5. The siRNA expression cassette of claim 1 which expresses small RNA in mammalian cells.

6. The siRNA expression cassette of claim 2, wherein the indicator of cellular delivery is selected from the group consisting of a fluorescent dye, a quantum dot and an imaging molecule for non-invasive diagnosis.

7. The siRNA expression cassette of claim 6, wherein the non-invasive diagnosis is selected from the group consisting of NMR, MRI, CT, and PET scanner.

8. The siRNA expression cassette of claim 1, wherein the sequence encoding a nuclear delivery indicator is a sequence for GFP or luciferase operably linked to a promoter.

9. The siRNA of claim 8, wherein the promoter is tissue specific.

10. A kit for in vitro and/or in vivo gene knock down studies at RNA level which comprises the following parts:
1) a short interfering RNA (siRNA) expression cassette;
2) a reporter gene cassette that exhibits reporter gene expression if it is successfully delivered to, and expressed in, the nuclei of a eukaryotic cell; and
3) a transfection reagent that is able to efficiently deliver the siRNA expression cassette and the reporter gene cassette into the eukaryotic cells.

11. The kit of claim 10, wherein the siRNA expression cassette and the reporter gene cassette are on the same plasmid or vector.

12. The kit of claim 10, wherein the siRNA expression cassette further comprises an intracellular traffic marker.

13. The kit of claim 10, wherein the siRNA expression cassette is driven by a U6 promoter or a H1 promoter and expresses small RNA in mammalian cells.

14. The kit of claim 10, wherein the siRNA expression cassette expression is selected from the group consisting of non-specific expression, tissue-specific expression and cell-specific expression.

15. The kit of claim 12, wherein the intracellular traffic marker is selected from the group consisting of a fluorescent dye, a quantum dot and an imaging molecule for non-invasive diagnosis.

16. The kit of claim 15, wherein the non-invasive diagnosis is selected from the group consisting of NMR, MRI, CT, and PET scanner.

17. The kit of claim 10, wherein the reporter gene cassette is a fluorescent protein gene expression cassette operably linked to a promoter.

18. The kit of claim 17, wherein the promoter confers tissue specific expression.

19. The kit according to claim 17, wherein the fluorescent protein gene expression cassette comprises a green fluorescent protein gene.

20. The kit of claim 10, wherein the reporter gene cassette is adjacent to a DNA fragment of the siRNA expression cassette.

21. The kit of claim 10, wherein the interference RNA expression cassette and the report gene synthesis cassette are circular or linear.

22. The kit of claim 10, wherein the transfection agent is selected from the group consisting of lipid based transfection reagents with or without cationic groups, polymer based transfection reagents with or without cationic groups, lipid-polymer-based transfection reagents with or without cationic groups, polysaccharide-based transfection reagents with or without cationic groups, and peptide-based transfection reagents with or without cationic groups.

23. The kit of claim 10, wherein the transfection reagent further comprises a cell culture reagent.

24. The kit of claim 23, wherein the cell culture reagent is selected from the group consisting of cytoreductive reagents, cell attach reagents, cell growing reagents, and cell inhibiting reagents.

25. The kit of claim 10, wherein the transfection reagent is a gene delivery enhancer or a targeting molecule.

26. The kit of claim 25, wherein the gene delivery enhancer is selected from the group consisting of an endosomalytic reagent and a nuclear localization element.

27. The kit of claim 25, wherein the targeting molecule is selected from the group consisting of a peptide, a protein, an antibody or its related fragment, a sugar, and a synthetic molecule.
